(19) United States
(12) Patent Application Publication
(10) Pub. No.: US 2002/0033184 A1
(43) Pub. Date: Mar. 21, 2002

(54) Method and kit for removing aldehyde-based stains

(76) Inventors: Peter Zhu, Irvine, CA (US); Henry Hui, Laguna Niguel, CA (US); Les Feldman, Calabasas Hills, CA (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 09/894,402

(22) Filed: Jun. 28, 2001

Related U.S. Application Data
(63) Continuation-in-part of application No. 09/747,230, filed on Dec. 22, 2000.
Application No. 09/747,230 is a continuation-in-part of application No. 09/321,964, filed on May 28, 1999.

(51) Int. Cl. B08B 3/00; B08B 7/00; A62D 3/00; C09K 3/22; A61K 7/50; C03C 23/00; B08B 9/00; C23G 5/00; C11D 1/00; C23G 1/02; C23G 1/00

(52) U.S. Cl. 134/2; 588/218; 134/3; 134/22.1; 134/22.13; 134/22.17; 134/36; 134/40; 134/41; 510/130; 134/161; 510/214; 510/217

(57) ABSTRACT

A method and kit are disclosed for removing or alleviating the severity or intensity of stains from aldehyde-based sterilizing solutions through the use of reducing agents. This invention is effective even for porous surfaces such as filter paper and porous nylon films as well as for smooth surfaces such as glass, aluminum, copper, brass and stainless steel. Also, the invention may be used in a variety of formats such as a kit useful for making or using solutions or gels to treat the aldehyde-based stained surfaces of, for example, medical devices, hospital bench or table tops, hospital floors and skin (if in appropriate pH range), etc.

This patent application is a continuation-in-part of co-pending and commonly assigned patent application U.S. Ser. No. 09/747,230, filed Dec. 22, 2000 entitled “Reductive Amination for Aldehyde Neutralization Related Applications” which is a continuation-in-part of co-pending and commonly assigned patent application U.S. Ser. No. 09/321,964, filed May 28, 1999 entitled “Aldehyde Neutralizer”, the disclosures of which are hereby incorporated by reference.

### BACKGROUND OF THE INVENTION

1. Field of the Invention

The invention relates to a method and kit for removing or lessening the severity of aldehyde-based stains, in particular, stains formed by reaction of aldehydes with amino-containing compounds.

2. Description of Related Art

Aldehyde-based germicides are commonly used in the healthcare facilities to disinfect and sterilize medical devices. One related problem associated with the use of aldehyde-based germicides such as formaldehyde, glutaraldehyde and ortho-phthalaldehyde (OPA) that have been reacted with amino-containing compounds is that the surfaces and/or the material that come into contact with the neutralized aldehyde become stained. Also residual aldehyde on surfaces or absorbed into the materials such as plastic will cause staining later when exposed to amino-containing compounds. This is a problem in a hospital enviromnent when sterile surfaces and/or materials that are stained can cause concern that the devices are not sterile. Such surfaces include but or not limited to bench tops, floor surfaces, medical devices (metal or plastic) and human skin. Some OPA related stains are the results of formation of Schiff's bases which are dark in color. Although the staining in the hospital environment is very problematic, there are no good measures to solve the problem at present. This invention offers solutions to this problem.

### SUMMARY OF THE INVENTION

In one embodiment the invention relates to a method for removing or lessening the severity of aldehyde-based stains on surfaces or in materials comprising the steps of:

a) providing an aldehyde-based stain on a surface or in a material; and

b) applying a reducing agent to the stained surface or material.

In another embodiment, the invention relates to a kit comprising:

a) a source of reducing agent; and

b) instructions regarding the proper amount of reducing agent to be combined with a solvent or a gel in order to provide a solution or a suspension of effective strength to remove or lessen aldehyde-based stains.

Among the advantages of the invention is removal of aldehyde-based stains to no stain or at least to a stain of lessened severity or intensity. This invention is effective even for porous surfaces such as filter paper and porous nylon films as well as for smooth surfaces such as glass, aluminum, copper, brass and stainless steel. Also, the invention may be used in a variety of formats such as a kit useful for making or using solutions or gels.

Additional features, embodiments, and benefits of the invention will be evident in view of the detailed description presented below.

### DETAILED DESCRIPTION OF THE INVENTION

Commonly assigned and co-pending patent application U.S. Ser. No. 09/321,964 discloses the neutralization of aldehdye with amino acid. The neutralized aldehyde product can be formed by reacting an amino group from an amino acid or proteins with an aldehyde group of aldehydes to produce N-substituted adducts (imines or Schiff's bases) as shown below.

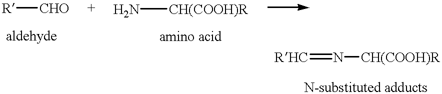

There are a variety of amino acids that are useful in neutralizing aldehydes. These amino acids include:

(1) Amino acids with apolar R groups (e.g., alanine, proline, amino-caproic acid, phenylalanine, tryptophan and methionine);

(2) Amino acids with polar R groups (e.g., glycine, serine, cysteine, tyrosine, and glutamine);

(3) Amino acids with charged R groups (e.g., aspartic acid, glutamic acid, lysine, arginine, and histidine); and,

(4) Peptides/polypeptides formed by any number or any type of amino acids and proteins.

The aldehyde can be a germicide that includes formaldehyde, glutaraldehyde, and OPA.

While the discovery of forming neutralized aldehydes for lessening the toxic effects of disposing of aldehyde treated waste was a major advance, resulting colored products (and hence susceptibility to forming stains) and the possibility of the neutralized aldehydes in reforming aldehydes under acidic conditions posed a problem in effectively maintaining nonhazardous waste because of the toxic effects of unneutralized aldehyde. The reversible reaction is depicted below for treatment of glutaraldehyde (1) and o-phthaladehyde (4) with the amino acid, glycine (2) to the neutralized products, (3) and (5), respectively:

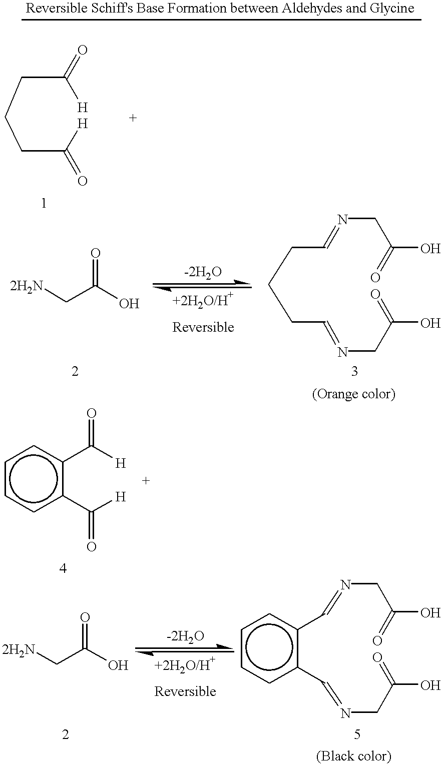

Commonly assigned and co-pending patent application U.S. Ser. No. 09/747,230 disclosed that the treatment of the neutralized products with a reducing agent to form amino acids do not cause them to revert back to unneutralized aldehyde. This reaction is depicted below for saturated moieties (6) and (7) as for the reduction of Schiff's bases (3) and (5) treated with the reducing agent sodium borohydride,

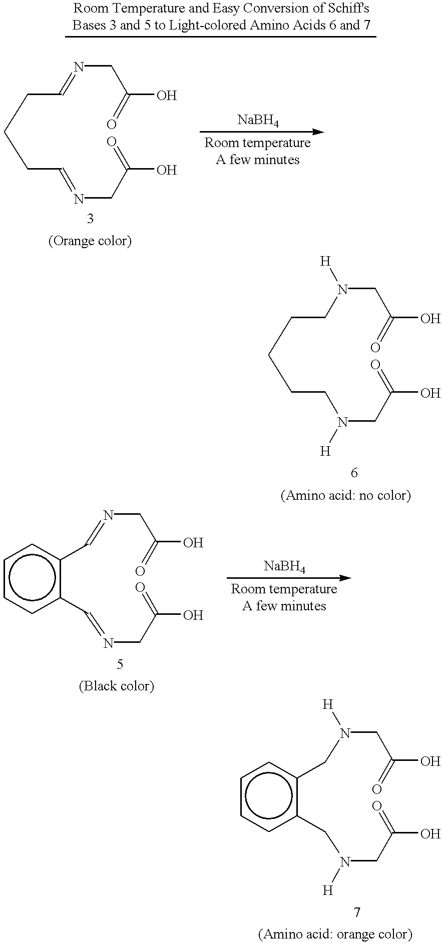

Being simple amino acids compounds (6) and (7) would be expected to be biodegradable and thus have significant benefit for the environment. This appears apparent by examination of the resemblance of the structures (6) and (7) with the natural essential amino acid proline, (8). The corresponding resemblance is depicted with bold-faced highlighting of compounds (6) and (7) shown below:

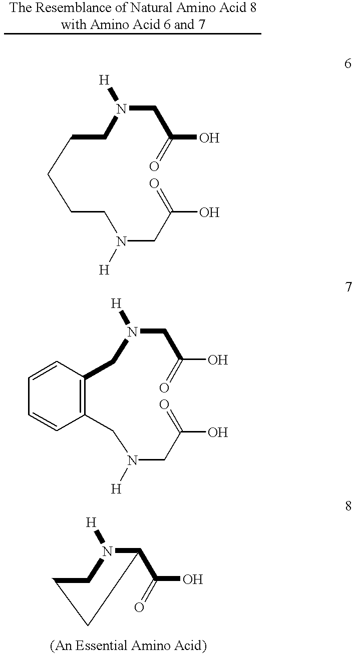

In contrast, Schiff's base (3) and (5) do not have the above characteristics and are likely very different compounds. One skilled in the art would suspect Schiff's bases to be harder to degrade in nature than the corresponding amino acids. For example, a piece of animal skin could decay in a few days in the wild while men's belts, made from animal skin too, could take many years. This is because the belt (leather) has undergone a tanning process. Tanning processes often employ the glutaraldehyde derivatives, such as depicted as structures (9) and (10) below, to cross-link proteins (Ref. a. Fein, M. L. and Filachione, E. M., “Tanning studies with aldehydes”, J. Am. Leather Chem. Assoc., 52, 17, 1957; b. Weligsberger, L. and Sadlier, C., “New developments in tanning with aldehydes”, J. Am. Leather Chem. Assoc., 52, 2, 1957; c. Hopwood, D., “Comparison of crosslinking abilities of glutaraldehyde, formaldehyde, and α-hydroxyadipaldehyde with bovine serum albumin and casein”, Histochemie, 17, 151, 1969). It is well known that OPA has very similar protein cross-liking properties.

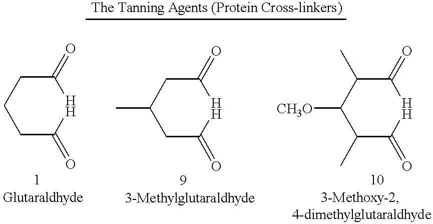

The conditions for Schiff's base reduction (and hence the removal or lessening of stains) is easy and convenient. Normally, it involves the mixing of the reducing agent, such as NaBH₄, and the imine, such as neutralized aldehyde, in a protonic solvent, such as water, ethanol, methanol, acid, or gel at room temperature. The acid may be organic or inorganic having active hydrogens. Due to the formation of colorless or light-colored products between the reducing agent and the imine, it is found that the reducing agent is a good chemical for removing the stains. The reducing agent can be applied to the stains by washing, spraying or dropping a solution containing the reducing agent onto the stained surface, by soaking the stained material in the solution containing the reducing agent, or by rubbing a gel or lotion comprising the reducing agent onto the stained surface or into the stained material.

The imine can be reduced by many reducing agents, such as LiAlH₄ (lithium aluminum hydride), NaBH4 (sodium borohydride), NaCNBH₃ (sodium cyanoborohydride), Na-EtOH (metallic sodium in ethyl alcohol), and H₂/catalyst (hydrogen with a catalyst). A preferred reducing agent is NaBH₄.

### EXAMPLES

In the following examples, all percentages (%) referring to solutions are expressed as wt/vol, except in the case of Cidex® OPA where the percentages is expressed as wt/wt. The pH of the 1% aqueous sodium borohydride solution used in the following examples was measured to be 10.8.

### Example 1

(a) Cidex® OPA solution (0.55%, 1.0 mL) and glycine solution (1%, 1.0 mL) were mixed in a vial for 2 minutes and a green color developed and darkened quickly.

(b) Sodium borohydride solution (1%, 1.0 mL) was added, shaken and mixed to the solution. The solution gradually became yellow and never darkened. The solution never turned black. Thus, the severity or intensity of the staining solution of (a) was lessened by the reducing agent sodium borohydride.

### Example 2

(a) Cidex® OPA solution (0.55%, 0.05 mL) was applied to porous nylon film for about 10 minutes at room temperature (until dry). Glycine (1%, 0.05 mL) was applied on the top of the Cidex® OPA spot. In about 10 minutes, the spot showed a green-black color.

(b) Sodium borohydride solution (1%, 0.15 mL) was applied onto the top of the green-black spot. The green-black spot was de-stained immediately to turn to a much lighter color (yellowish brown).

### Example 3

(a) Cidex® OPA (0.55%, 0.05 mL) was applied on porous nylon film and dried in about 2 hours at room temperature. Glycine (1%, 0.05 mL) was added on the top of the Cidex® OPA spot and after 2 minutes, the spot became green-black.

(b) The stained nylon film was then soaked in sodium borohydride solution (1%, 10 mL) for 10 minutes, the green-black spot changed to a light-yellow spot. The color was noted to continue to fade with continued soaking.

(c) For comparison, the same experiment was repeated. However, instead of soaking in the sodium borohydride solution, the film was soaked in the water for 10 minutes. No reduction of the green-black color was observed. In fact, the green-black color never faded by soaking in water for 2 days.

### Example 4

(a) Cidex® OPA solution (0.55%, 0.05 mL) was applied to filter paper (“Fisherbrand”, qualitative P2, porosity: fine) and dried at room temperature for two hours. Glycine (1%, 0.05 mL) was added on the top of the Cidex® OPA spot and after about 2 minutes, the spot became a green-black color.

(b) The stained filter paper was soaked in sodium borohydride solution (1%, 10 mL) for 5 minutes when the green-black spot changed to very light-yellow spot. The spot became colorless when the soaking continued for 2 hours.

(c) For comparison, the same experiment was repeated. However, instead of soaking in sodium borohydride solution, the filter paper was soaked in the water for 10 minutes. No reduction of the green-black color was observed. In fact, the green-black color never faded even after soaking in water for 2 days.

### Example 5

(a) Cidex® OPA solution (0.55%, 0.05 mL) was applied to two (2) aluminum coupons (1×1 inches) and glycine (1%, 0.05 mL) was added on the top of the Cidex® OPA spots. The aluminum coupons were then heated at 80° C. for 2 hours. A brown-black residual spot formed on each coupon.

(b) One coupon was then soaked in sodium borohydride solution (1%, 10 mL) at room temperature. Surprisingly, the black residue on the aluminum surface disappeared instantly (less than 3 seconds). The solution became clear yellow. The surprisingly good result on the metal surface was not expected.

(c) The other coupon, the control coupon, was soaked in 10 mL water, where most of the brown-black residue dissolved gradually. However, there was still residue left on the coupon after 3 hours. The solution became dark green with black residue deposit on the bottom of the vial.

### Example 6

Example 5 was duplicated with brass coupons with even better results. The brown-black residue came off instantly while that on the control coupon came off only partially and the remainder came off very slowly and it remained even after soaking in water for 2 days.

### Example 7

Example 5 was duplicated with copper coupons. The same excellent result was observed as in Example 5.

### Example 8

Example 5 was duplicated with stainless steel surfaces. Thee same excellent result was observed as in Example 5.

### Example 9

(a) Cidex® OPA solution (0.55%, 0.05 mL) was added into a scintillation vial and glycine (1%, 0.05 mL) was added and mixed. The solution was heated at 80° C. for 1 hour and a black residue was left on the bottom of the vial.

(b) Sodium borohydride solution (1%, 2 mL) was then added and the black residue was dissolved instantly to give a clear yellowish-brown solution (no green or black color).

(c) On the other hand, for the control experiment, the same procedure as above was followed except water (instead of sodium borohydride solution) was added to the vial. The black residue came off the glass wall after shaking and most of the residues remain suspended in the solution for 2 days. The solution appeared to be dark-green in color.

### Example 10

Examples 5-9 were repeated. In this case, fresh sodium borohydride versus aged solution (1 day old) was compared. All gave good results. All the black Schiff's base residue on different metal coupons came off instantly after exposing to the 1% sodium borohydride solution.

### Example 11

(a) Silicone tubing (OD=0.60 cm, wall thickness=2 mm) was soaked in the Cidex® OPA solution for seven days, washed with detergent, rinsed with water and then soaked in an amino solution to pick up the homogenous dark stains.

(b) The above silicone tubing (1.0 cm in length) was soaked in sodium borohydride (1%, 5 mL) for 2 hours, the majority of the color disappeared.

(c) Comparatively, the tubing soaked in water in place of the sodium borohydride did not fade in 1 month.

Therefore, based on Examples 1-11, it would be appreciated by those skilled in the art that depending on the nature of the material, the time or the efficiency to remove the stain may vary. Metal may require less time than non-metal, and absorbent or porous materials may require longer time than non-absorbent or non-porous materials.

### Example 12

Example 11 was duplicated with a slight modification. Instead of water, 50% ethanol in water was used as the solvent. In this case, compared to the control (water as solvent), the de-staining rate is somewhat faster.

### Example 13

(a) Procedure: Cidex® OPA solution (0.55%, 10 mL) was mixed with glycine (1%, 10 mL). The mixed solution separately was applied to brass and aluminum coupons (20 μL mixed solution for each stain). The coupons were then heated at 80° C. for 2 hours. Brown-black residual spots formed.

(b) Each coupon (with one stain spot on it) was soaked in aqueous NaBH₄ solutions of varying concentrations as shown in Table 1. The time it took to remove the stain spot completely was recorded in Table 1.

TABLE 1
Effect of concentration on the Time (in seconds) required to remove Schiff
based stains

| NaBH₄ | | | | | |
|---|---|---|---|---|---|
| Concentration | 1% | 0.5% | 0.25% | 0.125% | 0.0625% |
| Schiff' base stain on | 2” ± 0.5 | 3” ± 1 | 4” ± 1 | 15” ± 5 | 26” ± 7 |
| brass | | | | | |
| Schiff' base stain on | 3” ± 0.5 | 3” + 1 | 4” + 1 | 12” ± 5 | 22” ± 7 |
| aluminum | | | | | |

Thus aqueous NaBH₄ solutions of various concentrations are quite effective for rapid removal of Schiff-based stains

### Example 14

Reduction to product formats.

The following solutions could be used for soaking medical devices or wiping, or spraying medical surfaces or bench tops, floor surfaces etc. Since the pH is around 10.8, normal care should be taken (gloves/ventilation).

Reduction to product format—(14-1):

Sodium borohydride powder (0.5-5 g) is packed in bottles. Cap well. Add water before use (to dilute to near 1%).

Reduction to product format—(14-2):

Sodium borohydride powder (0.5-5 g) is packed in bottles. Cap well. Add mixture of water or an alcohol (ethanol, or isopropanol or methanol or mixed alcohol) before use (to dilute to near 1%).

Reduction to product format—(14-3):

Sodium borohydride powder (0.5-5 g) and an amino acid (such as glycine, lysine etc) are packed in bottles as dry powder. Cap well. Add mixture of water or an alcohol (ethanol, or isopropanol or methanol or mixed alcohol) before use (to dilute to near 1%).

Reduction to product format—(14-4):

A container in the form of a spray bottle or bottle with a dropper may be used for formats 14-1 to 14-3.

Reduction to product format—(14-5):

Instead of using bottle container, the sodium borohydride powder could be packed into aluminum bags or other types of bags to reduce product sizes.

Reduction to product format—(14-6):

Nitrogen or another inert gas could be filled into the packaging formats in order to increase shelf life.

Reduction to product format—(14-7):

Desiccators could be used for the above packaging formats in order to increase shelf life.

Reduction to product format—(14-8):

Other reducing agents of similar nature could be used, such as sodium cyanoborohydride, metallic sodium in ethyl alcohol, and H₂/catalyst.

The invention is well suited for formation of kits wherein the dry powder form of the reducing agent may be combined with a solvent such as water, alcohol, or mixtures thereof, to form a solution of desired concentration and pH. Instructions within the kit would provide guidance regarding the proper amount of reducing agent to be combined with the solvent in order to provide a solution of effective strength to remove or lessen aldehyde-based stains. The resulting solutions may be used as soaking, wiping, or spraying solutions to treat the stained surfaces, for example, of medical devices, bench or table tops, floors and skin (if in appropriate pH range), etc.

In the preceding detailed description, the invention is described with reference to specific embodiments thereof. It will, however, be evident that various modifications and changes may be made thereto without departing from the broader spirit and scope of the invention as set forth in the claims. The specification and drawings are, accordingly, to be regarded in an illustrative rather than a restrictive sense.

What is claimed is:

1. A method for removing or lessening the severity of aldehyde-based stains comprising the steps of:
a) providing an aldehyde-based stain on a surface or in a material; and
b) applying a reducing agent to the stained surface or material.

2. The method of claims 1, wherein the reducing agent is selected from the group consisting of LiAlH₄, NaBH₄, NaCNBH₃, Na-Ethyl alcohol, and hydrogen/catalyst and mixtures thereof.

3. The method of claim 1, wherein the aldehyde-based stain comprises an imine formed from an aldehyde and a chemical containing amino group.

4. The method of claim 3, wherein the aldehyde comprises a germicide.

5. The method of claim 3, wherein the aldehyde comprises one or more (—CHO) functional group.

6. The method of claim 3, wherein the aldehyde comprises a dialdehyde.

7. The method of claim 3, wherein the aldehyde is selected from the group consisting of ortho-phthalaldehyde, glutaraldehyde, formaldehyde and mixtures thereof.

8. The method of claim 3, wherein the chemical is selected from the group consisting of alkyl amine, aryl amine, polymer amine, protein, peptide, amino acid, and any combination thereof.

9. The method of claim 1, wherein the surface or material is selected from the group consisting of a bench top, floor, medical device, drape, gown, dressing and human skin.

10. A kit for removing or lessening the severity of aldehyde-based stains comprising:
a) a source of reducing agent; and
b) instructions regarding the proper amount of reducing agent to be combined with a solvent or a gel in order to provide a solution or a suspension of effective strength to remove or lessen aldehyde-based stains.

11. The kit of claim 10, further comprising a container within which to combine the reducing agent and solvent.

12. The kit of claim 10 or 11, wherein the instructions require use of a solvent selected from the group consisting of water, alcohol, acid, gel and mixtures thereof.

13. The kit of claim 12, wherein the acid includes organic and inorganic compounds with at least one active hydrogen.

14. The kit of claim 10, wherein the reducing agent is selected from the group consisting of LiAlH4, NaBH₄, NaCNBH₃, Na-Ethyl alcohol, and hydrogen/catalyst and mixtures thereof.

15. The kit of claim 11 wherein the container is a spray bottle.

16. The kit of claim 11 wherein the container comprises a dropper.

17. The kit of claim 11 wherein the aldehyde-based stain comprises an imine formed from an aldehyde and a chemical containing amino group.
